(19) United States
(12) Patent Application Publication
(10) Pub. No.: US 2018/0055593 A1
(43) Pub. Date: Mar. 1, 2018

(54) TWO-TONED FLUOROSCOPY DRAPES FOR ORTHOPAEDIC FRACTURE PROCEDURES

(71) Applicant: Daniel Robert Schlatterer, Dunwoody, GA (US)

(72) Inventor: Daniel Robert Schlatterer, Dunwoody, GA (US)

(21) Appl. No.: 15/246,051

(22) Filed: Aug. 24, 2016

(51) Int. Cl. A61B 46/00; A61B 90/00; A61B 46/10

(52) U.S. Cl. CPC A61B 46/40; A61B 2090/081; A61B 46/10; A61B 90/08

(57) ABSTRACT

The present invention is an improved method of maintaining sterility in the operating room during orthopaedic fracture care. This invention aids in the identification of when a non-sterile drape area has been touched by operating room personnel. This invention is a two-toned drape which is otherwise similar to a standard drape, but with contrasting or bright colors added to one half of the drape, and optionally to an outer 2 inch border to create two or more distinct and separate halves to the drape.

### TECHNICAL FIELD

The present invention relates to an improved method of maintaining sterility in the operating room. This invention also relates to aiding in the identification of when a non-sterile area has been touched by operating room personnel. One such application would be for all surgical drapes in a sterile procedure to be two-toned with a distinct visual marker.

### BACKGROUND OF THE INVENTION

Prevention of an infection during surgical procedures is of utmost importance. Surgical site infections (SSIs) are currently a major burden in health care. SSIs lead to increased length of stay for a given hospital admission for a given surgical procedure, increased costs relate directly to treating the infection itself and in some cases a SSI may lead to loss of a limb and even loss of a life.

Multiple protocols have been developed over the past century to reduce SSI rates. These protocols include; administration of antibiotics before the surgery begins, cleaning and cleansing the skin where the site of the incision is to be made, another protocol is creating a sterile field for the surgical procedure. Sometimes simply placing sterile sheets over a patient and the operating room table is not enough to create and maintain a sterile field, imagine a picnic table as an operating room table. In order to fully cover a large picnic table, it may require multiple sterile drapes and a small draft or wind gust may shift the drapes creating gaps and uncovered areas of the table. In order to reestablish full coverage of the picnic table, some part of each drape would need to be touched to allow moving the drape to a more covered position. In the process of the drapes shifting slightly, the drape edges likely touch non-sterile parts of the picnic table such as the sides or legs of the table or even the ground. Anyone on the OR team may compromise sterility of the entire surgical field if they inadvertently touch these areas with their gloved hands.

The same event sequence is at risk for the portable x-ray machine also known as the fluoroscopy machine or C-arm 120. The C-arm 120 is used to take radiographic images during an orthopaedic surgery. The machine must be brought very close to the patient in order to image their arm or their leg or any other body part. The C-arm must be draped to cover its non-sterile components. This draping process is repeated over and over as the C-arm is moved close to the patient and then away from the patient in order for the surgeon to continue his/her procedure. Then after a little bit of progress the C-arm 120 is brought back in to have another radiologic image taken to check the progress. It is this in and out of the C-arm 120 in which the machine is draped and undraped that field compromise occurs. Field compromise occurs because the surgical team does not have a good visual indication of where to touch the C-arm 120 drape and where not to touch. This invention takes an already commercially available drape and adds bright colors to certain areas of the drape to delineate no touch zones.

Creating and maintaining a sterile field is a labor intensive task requiring all operating personnel to wear sterile gloves and gowns. The operating room personnel must be diligent and carefully watch what they touch and where and what other members of the surgical team touch or what they come into contact with. It is a collective effort to keep watch on each other. Surgical team members alert each other if they observe that someone on the team has touched or come into contact with something not sterile. The contact is most often the C-arm 120 drape. These areas are very hard for the surgical team members to appreciate, and avoid contacting with their hands.

A surgical site infection follows when something laden with bacteria enters the surgical site. The sources of this bacteria are too numerous to list. Suffice it to say that at some point a break in sterility occurs, either a hole in the surgeon's glove or particles floating in the air settle into the surgical incision and an infection develops days later. Or a member of the surgical team touches something not sterile and carries foreign bacteria laden particles to the surgical site. Unfortunately, wearing gloves only protects the surgical team member's hands and fingers. Gloves often serve as vectors of foreign particles, and the only way to prevent transmitting something into the surgical site is to make sure the gloves never touch anything non-sterile, this includes previously sterile drapes. No drapes remain completely sterile for the entire duration of any surgical procedure, especially the drapes over a C-arm 120.

It is for these reasons that maintaining sterility in and around the surgical field is imperative. The surgical team cannot get holes in their gloves, lose their caps or masks, or contaminate their gowns, or touch anything not completely sterile. Contaminated gowns or drapes may contaminate the gloves of someone on the surgical team, these same gloves would then cross contaminate the sterile instruments and the sterile implants becoming vectors carrying bacteria into the surgical site.

Prevention is the key to reducing SSIs. Drapes with better visualization and color demarcations of high risk contamination areas would help reduce inadvertent glove contamination.

### SUMMARY OF THE INVENTION

The present invention is a surgical cloth or paper drape with two additional features: 1) one portion of the drape, preferably down the middle, would be a contrasting or bright color, either orange yellow or red, or any variation thereof which would include but not be limited to hash marks shading or various combination or singular design feature to form a visual marker. 2) These same two-toned drape areas, by example, one half of the drape down the middle, could have a resin painted on that would transfer to any object it comes into contact with. Resin transfer will result in a visible change in the drape causing a color change and/or bare spot in the resin loss region of the gown. The resin could be any non-toxic material such as charcoal.

The large scale problem is surgical site infections which is directly related to the smaller problem which is poor visibility of frequently contaminated areas of drapes, the solution can be twofold. The first solution is to manufacture a two-toned drape. The back of the drape would have a two-tone feature that would be brightly colored such as yellow, orange or red or pink. Color demarcations would better assist all people in the OR to identify areas of the drape that are absolutely no touch zones. The color demarcation would also make it easier for some member of the OR team to identify if this area came into contact with something not sterile. Someone in the OR could more easily say, “Yes, I just saw the orange part of the drape touch the wall”, “yes, you need to change the drape, and or your gloves”.

A second solution would be to cover these same colored areas of the drape with some sort of transferable resin. Whereby this resin would come off of the drape and mark whatever it comes into contact with whether it be the wall or another object or person's gloves in the Operating Room. This process would ensure identification of a break in sterility because there would be a bare spot on the drape or even a color change and help identify all persons and, or objects no longer sterile in the OR because these persons or objects would have drape residue on them signifying that something inadvertently touched them and broke sterility.

An improved C-arm fluoroscopy drape has color changes and border demarcations of currently available surgical drapes. The drape described herein may further include any dimensional changes length or width alterations to improve draping of the C-arm 120 during orthopaedic procedures. The C-arm drape may be further improved via the materials employed and/or the thickness of the drape to avoid tearing or puncturing of the drape.

### BRIEF DESCRIPTION OF THE DRAWINGS

The invention will be described by way of example and with reference to the accompanying drawings in which:

FIG. 1 is a photograph of a typical fluoroscopy machine with a C arm 120.

FIG. 2 is a depiction of a patient 2 positioned with the C arm 120 vertically over the spine.

FIG. 3 is a depiction of the patient 2 lying on his back.

FIG. 4 is a photograph showing the patient 2 lying on his back with the C arm 120 under the table similar to FIG. 3.

FIG. 5 is a photograph showing the surgeon 4 standing near the C arm 120 pinching the drape 200 with his left hand 12.

FIG. 6 is a photograph showing the surgeon 4 standing near the C arm 120 folding the drape 200. The drawstring tie on the surgeon's 4 left hip area and under his left elbow marks the level of the surgeon's 4 waist. All of the drape in the surgeon's 4 gown front and to the floor is considered non-sterile. This 2 foot×4 foot area should never be touched and it should never contact any drape or other gowns

FIG. 7 is a photograph showing the two-toned drape 200V of the present invention held with a clamp 202. The size of the orange colored drape area 201 roughly approximates the 2 foot×4 foot frontal area of all gown fronts from waist area to the floor.

FIG. 8 is a photograph showing the surgeon 4 preparing to reposition the two-toned drape 200V, 201. By folding the drape and clamping it to itself the C-arm can be backed away from the patient and then later on the drape can be unclamped pulled out to properly drape conceal and cover the C-arm 120 and reused for more imaging with the C-arm 120 as indicated.

FIG. 9 shows an exemplary two-toned drape. One half of the drape is standard issue blue the other half again approximately 2 foot×4 foot corresponds to high risk areas from the front of surgical gowns with a similar color demarcation around the border of the drape 200B. A further iteration envisions a standard blue drape with a 2 inch orange border on all sides of the drape and on one half of the drape with a 2 foot×4 foot orange area

### DETAILED DESCRIPTION OF THE INVENTION

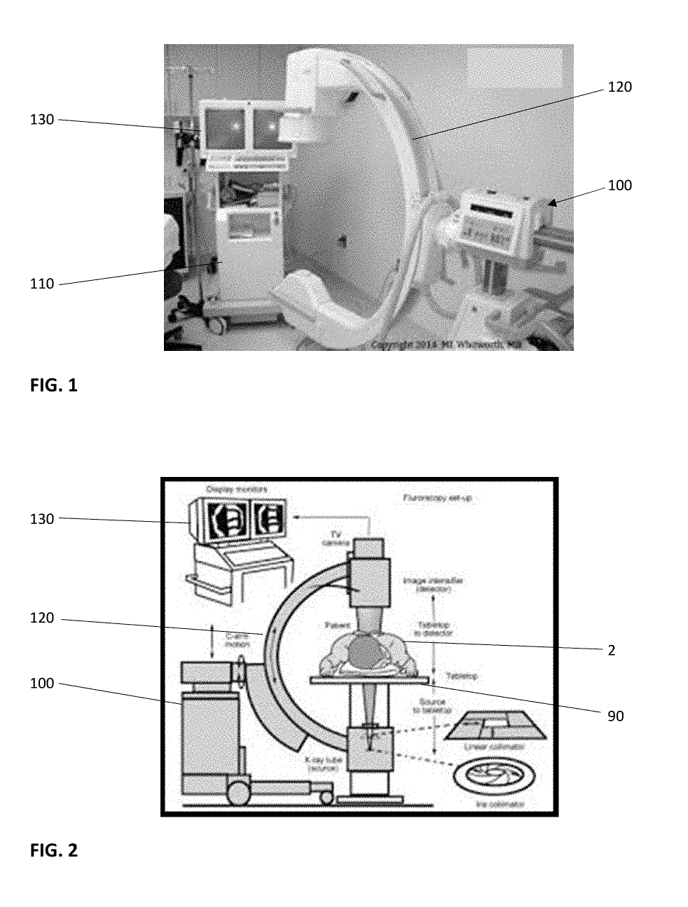

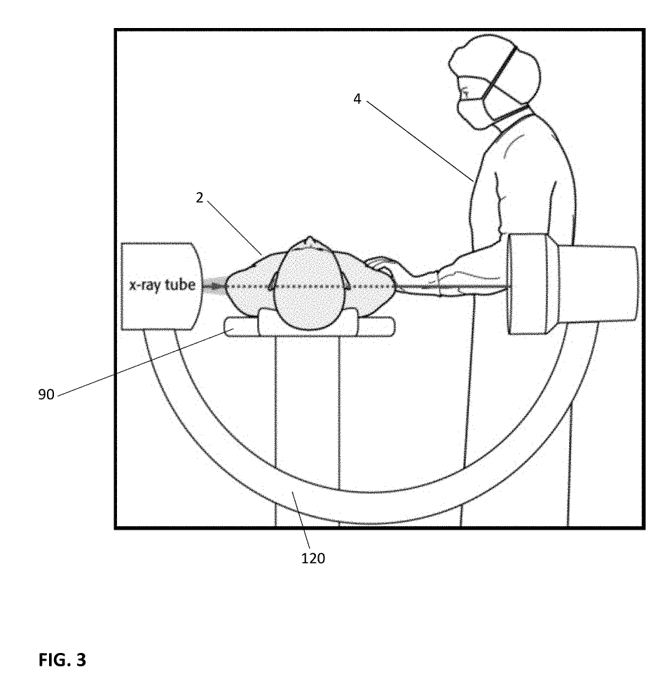

FIG. 1 is a photograph of a typical fluoroscopy machine 100. The machine 100 consists of two primary components the part which takes the actual radiographic image is in the right foreground 120,100. It has a large metal arm 120 in the shape of the letter C, often referred to in the operating room as the “C” arm 120, this cavernous shape permits the machine 100 to fit around a patient and acquire radiographic images as shown in FIGS. 2 and 3. The second component is a large cabinet 110 with side by side monitors 130 on top to project the radiographic images for immediate intra-operative viewing. There are no films to develop with the use of fluoroscopy; its onboard computer generates images to project on its monitors Both components 110, 120 are on wheels for easy repositioning in the operating room to acquire images of different body parts and in different projections, compare FIG. 2 versus FIG. 3. For example, the victim of a motorcycle collision may require orthopaedic procedures on both legs and sometimes even all four extremities. So the C-arm 120 is moved around quite a bit.

FIG. 2 shows a patient 2 positioned prone on their belly and the C-arm 120 is positioned vertically over their spine. Spinal images are shown on the fluoroscopy monitors 130.

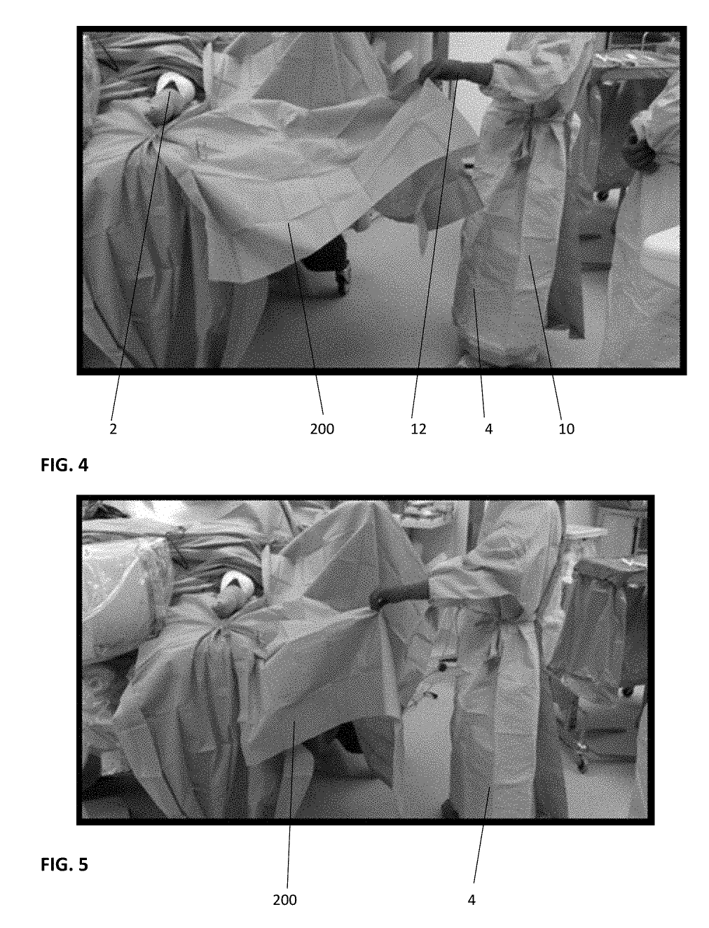

FIG. 3 shows the patient flat on his back and the C-arm 120 has been swung 90 degrees under the table 90 and the surgeon 4 is attempting to get a side-to-side radiographic image as opposed to the top-to-bottom projection seen in FIG. 2. Swinging the C-arm 120 around runs the risk of compromising the sterile field. Careful re-draping of the C-arm 120 prevents breaks in sterility. For example, in this figure, the entire left arm of the surgeon 4 is at risk of having non-sterile portions of the C-arm 120 touch him or her. Once sterility of the operative field is lost, the risk of a surgical site infection increases. The best way to prevent this infection risk is to carefully re-drape the C-arm 120 as it swings around, see FIGS. 4-8. The surgeon may have to change into a new gown and gloves. All of this activity increases the risk of field contamination and surgical site infections, and should be avoided at all costs.

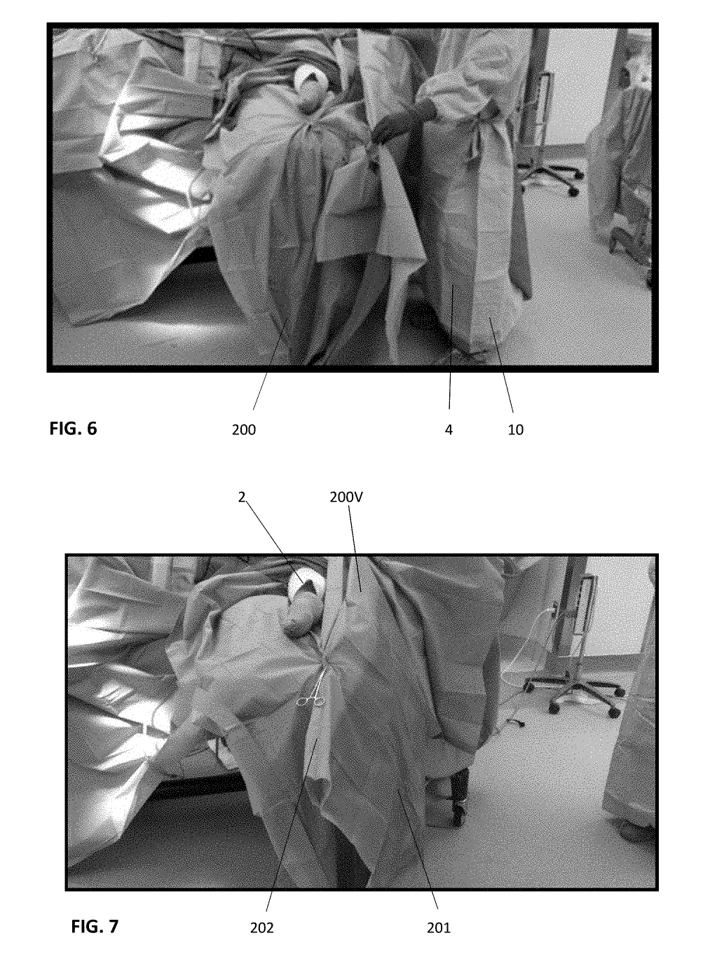

In FIG. 4, the patient is flat on his back again and the left arm is being operated on, note he white bandage around the extremity. The C-arm 120 was likely in a vertical position moments prior to this photograph similar in position to FIG. 2, and has just been swung under the table 90 degrees moving the C-arm 120 to a side-to-side position similar to FIG. 3. The surgeon 4 is standing to the right in this figure supporting a large blue drape 200 preventing the drape from touching his/her gown and from touching the floor. Directly in front of the surgeon 4 part of the C-arm 120 is tenting the new blue drape 200. Directly in front of his left hand 12 a clamp can be seen holding the drape in place for reuse again later in the procedure. Often this drape 200 is folded in half and secured by two clamps and then unclamped and unfolded to allow the C-arm 120 to re-enter the surgical field for imaging purposes, see drape 200 movement sequences in FIGS. 5, 6 and 7. This occurs as the C-arm 120 moves from a side-to-side horizontal position to a vertical position and then back to a side-to-side view. The vertical position affords a particular image to be taken and permits C-arm movement in towards the patient and out to image another extremity or allow the surgical team to continue working. The C-arm 120 often gets in the way of the surgical team so back and forth movements from horizontal to vertical and back again occur quite frequently. In addition the entire C-arm 120 is often backed all the way out of the operative field to enable the surgical team more direct access to the patient 2. Every series of C-arm 120 movements and repositioning's risks breaking the field sterility and increases SSI rates. Keep in mind that the surgeon's gown 10 in the back and everywhere below his or her waist is non-sterile. In FIG. 4, the drape 200 is too close to an area of the surgeon's gown 10 below his or her waist. So, in the next FIGS. 5 and 6, as the surgeon 4 steps forward and closer to the drape 200 he or she is holding a portion of the drape very near an area of their own gown which is no longer considered sterile. And either the drape 200 would have to be changed out or the surgeon 4 would have to put on a new gown 10 and gloves 12 as well to re-establish a sterile field, or the surgeon would only touch those areas of the drape 200 he or she is certain are still sterile. All of this activity increases the risk of surgical site infections (SSIs).

FIG. 5 is very similar to FIG. 4 except now the surgeon 4 is pinching the halfway point of the drape 200 with his left hand. See next sequences of drape movement in FIGS. 6 and 7. Keep in mind again that everywhere below the gown wearer's waist is non-sterile. The paper tie under the surgeon's 4 left elbow is at his/her waist level the drapes must be kept away from these gown areas which does not happen consistently. So in the next figures as the surgeon 4 in a gown 10 steps forward and closer to the drape 200 he or she is holding a portion of the drape 200 which is no longer sterile because it came close to or touched an area below the waist of the person in a gown 10 who is also handling the C-arm 120 drape 200. Two surfaces do not have to be in contact for a set period of time for cross contamination to occur. Two surfaces only have to touch for an instant for a break in sterility to be appreciated.

FIG. 6: same as FIGS. 4, and 5. This FIG. 6 is taken near the end point of folding the drape 200 in half and clamping the drape 200 for reuse later in the procedure. Reuse of this C-arm drape 200 would only require unclamping the drape and pulling it out and over the C-arm 120 as it came back around to a horizontal position from a vertical position.

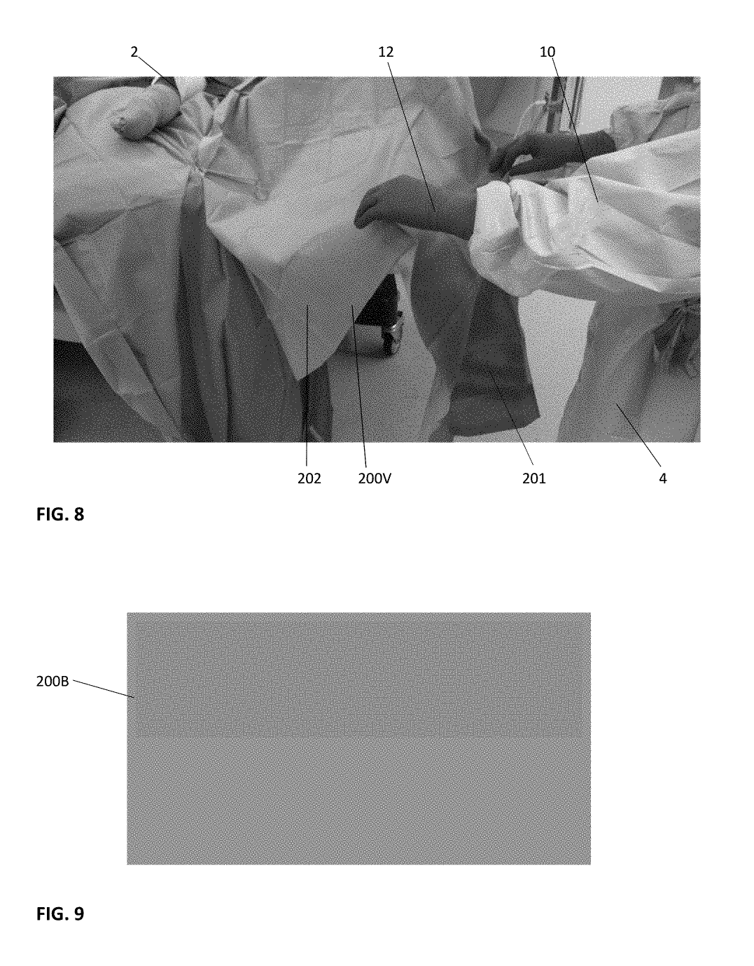

Maintaining a sterile field is especially challenging when there is a large machine moving in and out of the surgical field such as the C-arm 120. One of the drapes over the C-arm tends to slide off during its movement, at this point everyone reaches to keep the drape from reaching the floor. Some areas of this drape are perfectly fine to touch, but not the entire drape. Some areas, see FIGS. 4, 5, and 6, come far too close to the non-sterile front and lower portion of the surgeon's gown. The drape area most consistently compromised has been spray painted orange 201 in FIG. 7. In FIG. 8, the surgeon is mistakenly touching this area only for purposes of illustration. Seeing this colored area would be an easy visual for the surgical team members to avoid had this drape concept been available for this particular procedure. If and when need be anyone could grab this drape outside the orange areas, and avoid contaminating themselves.

In FIG. 7, the drape 200V has been spray painted orange on an area of 2 feet×4 feet one half or portion 201. This orange drape half or portion 201 is off limits to touching because when the drape was out and over the C-arm this orange area came too close to the front of the surgeon's gown (see FIG. 4), and it is now more obvious to anyone approaching this two colored drape 200V where to handle it and where to avoid touching it. Anyone touching within the orange area will likely have contaminated their gloves FIG. 7 is the end point of folding the drape 200V in half and re-clamping the drape 200V to the table 90 for reuse later in the procedure. The C-arm drape 200V, a few minutes earlier to taking this photograph, was spray painted orange on the lower half. No actual surgical procedure was happening during acquisition of these demonstrative photographs. The orange half of the drape is too close to the floor and too close to the front of the surgeon's gown 10 waist area and below, as shown in FIGS. 4 and 6. This orange half or portion 201 is frequently the area most often causing breaks in the sterility. These sterility breaks occur because this drape is commonly used in this fashion to drape the C-arm 120 and it almost always touches the floor or the front of the surgeon's gown 10 below his or her waist see previous FIGS. 4, 5, 6, and then members of the surgical team inadvertently touch this non-sterile drape area with their gloves.

The problem is that members of the surgical team often do not know this exact area is even compromised. The solution to this visibility problem is to manufacture a two-toned drape 200V that would easily demarcate the areas of the drape most frequently contaminated with a painted, dyed or otherwise visually marked area such as 201 in FIG. 7 not to be touched when folding and unfolding to drape and undrape the C-arm 120. This orange area 201 FIG. 7 represents the most commonly compromised area of the C-arm drape 200V. This orange area 201 would be a more visible off limit area to touching and it would be a more obvious area for the surgical team to avoid.

In FIG. 8, it is more obvious to the assistant or surgeon 4 that he/she is touching an off limit area 201 for touching on the C-arm drape 200V.

Fewer breaks in sterility would lead to fewer needless changes of the C-arm drape 200V and less gown 10 and glove 12 changes by the surgeon 4 to reestablish a sterile field. All of this decreased rates of breaks in sterility human movement and in turn decreased dander shedding activity in and around the surgical field because of a well demarcated C-arm drape 200V would lead to fewer breaks in sterility and less human movement due to fewer gown and glove changes and in turn decreased dander shedding, in turn, this would decrease the risk of surgical site infections with better patient outcomes after surgical procedures.

In FIG. 8, the surgeon 4 is about to reposition the C-arm drape 200V. Half of this drape 200V is orange in color 201. The surgeon 4 has smartly chosen to grab the C-arm drape 200V out of the orange colored area 201 with his or her left hand 12. Unfortunately the surgeon's right hand is right in the middle of the likely contaminated orange area 201. This hand positioning was done intentionally to illustrate the ease in which a colored area of a C-arm drape 200V is easy to identify and avoid touching when so instructed. Again, for the purpose of illustration, the entire purpose of this two-toned drape 200V at the moment of this photograph, the surgeon 4 was asked to use one hand to grab a non-orange area 202 of the drape 200V and to grab an orange area 201 of the drape with his or her other hand and that request was easily accomplished. If the instruction was to grab this drape with both hands, but avoid any contact with any orange area 201 to avoid contaminating his or her gloved hands, one could imagine similar ease of that task because of the bright orange color being readily visible.

FIG. 9 shows an exemplary drape similar to the half orange colored drape 200V in FIGS. 7 and 8, but in addition it would have an outer 2 inch border on the drape 200 B painted orange as well to demarcate no touch areas especially when repositioning drapes that have shifted for whatever reason.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

1. An improved operating room surgical drape comprises:
a two-toned surgical drape which is similar to a surgical drape with contrasting or bright colors added to evidence a non-sterile portion or half.

2. The improved operating surgical drape of claim 1 further comprises:
a contrasting or brightly colored outer border or perimeter portion.

3. The improved operating room surgical drape of claim 1 comprises:
drape areas having a transfer resin in the contrasting or bright color regions that would transfer from the drape and stain whatever it came into contact with in either a permanent or temporary fashion.

4. An improved C-arm fluoroscopy drape comprises:
a two-toned drape having one part of a base color and another part with contrasting or bright colors added to one half of the drape as a visual marker to indicate a non-sterile field.

5. The improved C-arm drape of claim 4 may also have an iteration whereby the two drape areas intended to be different colors by any manufacturing means with the end point of two or more well demarcated drape areas, these separate areas of the drape have the color difference areas substituted or combined with a resin that would transfer from the drape and stain whatever it came into contact with in either a permanent or temporary fashion.

6. The improved C-arm fluoroscopy drape of claim 4 further comprises:
demarcated areas for visually aiding in the identification of areas of the C-arm drape permissible for touching by members of the surgical team only.

7. An improved method of draping and undraping a C-arm for ease of repositioning the C-arm in an operating room wherein portions of a drape positioned in a sterile field are visually contrasted from portions of the drape outside the sterile field comprises the step of positioning a two-toned drape on the C-arm wherein one portion visually shows and represents a sterile field region using a contrasting color.

8. The improved operating room surgical drape of claim 1 wherein the contrasting colored areas of the drape may also be demarcated by any other distinguishing marks to include but not be limited to hash marks, dots or any other geographic shape, insignia trademark, logo, animal, figure or other inanimate object.
